(19) United States
(12) Patent Application Publication
(10) Pub. No.: US 2026/0360475 A1
(43) Pub. Date: Nov. 12, 2026

(54) ASCERTAINING THE STABILITY OF PACKAGED FORMULATIONS

(71) Applicant: Bayer Aktiengesellschaft, Leverkusen (DE)

(72) Inventors: Sebastian NIEDENFUEHR, Leverkusen (DE); Andreas WESTPHAL, Leverkusen (DE); Florian HAESE, Leverkusen (DE); Mauro STILLGER, Leverkusen (DE)

(21) Appl. No.: 19/111,940

(22) PCT Filed: Sep. 5, 2023
(86) PCT No.: PCT/EP2023/074224

§ 371 Date: Mar. 14, 2025

(30) Foreign Application Priority Data
Sep. 14, 2022 (EP) 22195615.4

(51) Int. Cl. G16C 20/30; G16C 20/70; G16C 60/00

(52) U.S. Cl. CPC G16C 20/30; G16C 20/70; G16C 60/00

(57) ABSTRACT

The invention concerns the process of ascertaining the stability of systems comprising a formulation and at least one material which is in contact with a formulation using a machine learning model. The invention relates to a method for training the machine learning model, to a computer-implemented method for predicting at least one stability characteristic of the system using the trained model, and to a computer system and a computer program product for carrying out the prediction method.

The present invention is concerned with ascertaining the stability of systems comprising a formulation and at least one material in contact with the formulation with the aid of a machine learning model. The present invention provides a method of training the machine learning model, a computer-implemented method of predicting at least one stability feature of the system by means of the trained model, and also a computer system and a computer program product for executing the prediction method.

Formulations are ubiquitous. We encounter them in the form of cosmetic products, pharmaceuticals, crop protection products and many other preparations, auxiliaries, intermediates and/or end products.

A formulation is produced from defined amounts of substances according to a defined recipe. A formulation is generally used to bring one or more constituents of the formulation into a form that fulfills an intended purpose. In the case of crop protection products (such as insecticides, fungicides and/or herbicides), one or more active ingredients together with formulation auxiliaries are generally converted to a form that improves the biological action of the active ingredient(s) in a target organism compared to the pure active ingredient(s) and/or makes the finished product usable for a defined purpose (e.g. deployment of a crop protection product by a particular application technique) and/or assures chemical stability of the active ingredient(s) under defined conditions. There are further purposes for which a formulation is created.

Formulations are stored and/or transported in various containers, where they come into contact with various materials. A question that arises in the case of newly developed formulations is which materials are suitable for a container or seal or the like for the formulation. It is conceivable that constituents of the formulation will damage a material in that they penetrate into the material and cause it to swell, for example. It is also conceivable that substances are transferred from a material (container, seal, etc.) into the formulation and contaminate the formulation and/or have adverse effects on the formulation.

Tests are typically conducted in order to check the suitability of materials for formulations. Many tests proceed by standardized methods. Such tests can be time-consuming and costly. Since such tests are often storage studies, the provision of a sufficient number of suitable storage options can also be a problem.

U. Siripatrawan and P. Jantawat disclose a method of predicting the shelf life of packaged moisture-sensitive snacks with the aid of a trained machine learning model (A novel method for shelf life prediction of a packaged moisture sensitive snack using multilayer perceptron neural network, Expert Systems with Applications, 34 (2008) 1562-1567). Shelf life is affected essentially by penetration of moisture into the packaging. The snacks are represented merely by generic constituents such as ash content, fat content, carbohydrate content and the like; the chemical compounds actually present in the snacks and their chemical and/or physical properties are not taken into account. Thus, although the method is suitable for predicting the shelf life of particular packaged moisture-sensitive snacks, it is impossible to apply the results to other formulations. For example, a prediction of what influence a formulation has on a material in contact with the formulation and/or what influence a material has on a formulation in contact with the material is impossible by this method.

The present invention is dedicated to this and other problems. The present invention provides a means by which at least one stability feature for a system comprising a formulation and at least one material in contact with the formulation can be predicted with the aid of a machine learning model.

The present invention firstly provides a computer-implemented method of training the machine learning model. The training method comprises the steps of:
providing and/or receiving information about a multitude of reference formulations, where the information for each reference formulation comprises (i) a recipe and (ii) at least one measured stability feature, where the stability feature is a measure of the stability of a system comprising the reference formulation and at least one material in contact with the reference formulation,
ascertaining constituents of the reference formulation on the basis of the recipe,
ascertaining physical and/or chemical properties of the ascertained constituents of each reference formulation,
creating a numerical representation for each reference formulation, where the numerical representation of the reference formulation comprises a numerical representation of the ascertained constituent for each ascertained constituent, where the numerical representation of each ascertained constituent comprises a value for the amount of the constituent in the reference formulation and values for the ascertained physical and/or chemical properties of the constituent,
training the machine learning model, where the training comprises the following steps for each reference formulation:
   inputting the numerical representation of the reference formulation into the machine learning model, where the machine learning model is configured to predict at least one stability feature based on the numerical representation of the reference formulation and model parameters,
   receiving the at least one predicted stability feature from the machine learning model,
   quantifying a variance between the at least one preferably measured stability feature and the at least one predicted stability feature,
   modifying the model parameters with regard to reducing the variance,
storing and/or outputting the trained machine learning model and/or transmitting the trained machine learning model to a separate computer system and/or using the trained machine learning model to predict at least one stability feature of a new system comprising a formulation and at least one material in contact with the formulation.

The present invention further provides a computer-implemented method of ascertaining at least one stability feature of a system comprising a test formulation and at least one material in contact with the test formulation by means of the trained machine learning model. The method comprises the steps of:
receiving a recipe of the test formulation,
ascertaining constituents of the test formulation based on the recipe of the test formulation,
ascertaining physical and/or chemical properties of the ascertained constituents,
creating a numerical representation of the test formulation, where the numerical representation of the test formulation comprises a numerical representation of the ascertained constituent for each ascertained constituent, where the numerical representation of each ascertained constituent comprises a value for the amount of the constituent in the test formulation and values for the ascertained physical and/or chemical properties of the constituent,
inputting the numerical representation of the test formulation into the trained machine learning model,
receiving at least one predicted stability feature from the trained machine learning model,
outputting and/or storing the at least one predicted stability feature and/or transmitting the at least one predicted stability feature to a separate computer system.

The present invention further provides a computer system comprising
an input unit,
a control and computation unit and
an output unit,
   wherein the control and computation unit is configured to
provide a trained machine learning model,
   wherein the trained machine learning model has been configured and trained with the aid of training data to predict at least one stability feature for a system comprising a formulation and at least one material in contact with the formulation based on a numerical representation of the formulation,
   wherein the training data for each reference formulation of a multitude of reference formulations comprise (i) a numerical representation of the reference formulation and (ii) at least one measured stability feature, where the at least one measured stability feature is a measure of the stability of a system comprising the reference formulation and at least one material in contact with the reference formulation,
   wherein the training of the machine learning model for each reference formulation comprises:
      inputting the numerical representation of the reference formulation into the machine learning model,
      receiving at least one predicted stability feature from the machine learning model,
      quantifying a variance between the at least one measured stability feature and the at least one predicted stability feature,
      modifying the model parameters with regard to reducing the variance,
cause the input unit to receive a recipe of a test formulation,
ascertain constituents of the test formulation on the basis of the recipe,
ascertain physical and/or chemical properties of the ascertained constituents,
create a numerical representation of the test formulation, where the numerical representation of the test formulation comprises a numerical representation of the ascertained constituent for each ascertained constituent, where the numerical representation of each ascertained constituent comprises a value for the amount of the constituent in the test formulation and values for the ascertained physical and/or chemical properties of the constituent,
input the numerical representation of the test formulation into the trained machine learning model,
receive at least one predicted stability feature from the trained machine learning model,
cause the output unit to output the at least one predicted stability feature and/or to store and/or to transmit it to a separate computer system.

The present invention further provides a computer-readable storage medium comprising commands which, on execution by a computer system, cause the latter to execute the following steps:
providing a trained machine learning model,
   wherein the trained machine learning model has been configured and trained with the aid of training data to predict at least one stability feature for a system comprising a formulation and at least one material in contact with the formulation based on a numerical representation of the formulation,
   wherein the training data for each reference formulation of a multitude of reference formulations comprise (i) a numerical representation of the reference formulation and (ii) at least one measured stability feature, where the at least one measured stability feature is a measure of the stability of a system comprising the reference formulation and at least one material in contact with the reference formulation,
   wherein the training of the machine learning model for each reference formulation comprises:
      inputting the numerical representation of the reference formulation into the machine learning model,
      receiving at least one predicted stability feature from the machine learning model,
      quantifying a variance between the at least one measured stability feature and the at least one predicted stability feature,
      modifying the model parameters with regard to reducing the variance,
receiving a recipe of a test formulation,
ascertaining constituents of the test formulation on the basis of the recipe,
ascertaining physical and/or chemical properties of the ascertained constituents,
creating a numerical representation of the test formulation, where the numerical representation of the test formulation comprises a numerical representation of the ascertained constituent for each ascertained constituent, where the numerical representation of each ascertained constituent comprises a value for the amount of the constituent in the test formulation and values for the ascertained physical and/or chemical properties of the constituent,
inputting the numerical representation of the test formulation into the trained machine learning model,
receiving at least one predicted stability feature from the trained machine learning model,
outputting and/or storing the at least one predicted stability feature and/or transmitting the at least one predicted stability feature to a separate computer system.

Further subjects of the present invention and preferred embodiments can be found in the dependent claims, the present description and in the drawings.

The invention is more particularly elucidated hereinafter without distinguishing between the subjects of the invention (training method, prediction method, computer system, computer-readable storage medium). Instead, the elucidations that follow are intended to apply analogously to all the subjects of the invention, irrespective of the context (training method, prediction method, computer system, computer-readable storage medium) in which they are given.

Where steps are mentioned in a sequence in the present description or in the claims, this does not necessarily mean that the invention is limited to the sequence mentioned. Instead, it is possible that the steps are also executed in a different sequence or else in parallel with one another, unless one step builds on another step, which makes it imperative that the step building on the previous step be executed next (although this will become clear in the individual case). The sequences mentioned thus constitute preferred embodiments of the invention.

In certain places, the invention will be more particularly elucidated with reference to drawings. The drawings show specific embodiments having specific features and combinations of features, which are intended primarily for illustrative purposes; the invention should not be considered to be limited to the features and combinations of features shown in the drawings. Furthermore, statements made in the description of the drawings in relation to features and combinations of features are intended to be generally applicable, i.e. applicable to other embodiments as well and not limited to the embodiments shown.

The present disclosure describes means of predicting at least one stability feature of a system comprising a formulation and at least one material in contact with the formulation with the aid of a machine learning model.

The formulation is preferably a formulation of one or more crop protection products. What is meant by the term “crop protection product” is a composition used to protect plants or plant products from harmful organisms or to prevent the effect thereof, to destroy unwanted plants or parts of plants, to inhibit unwanted growth of plants or to prevent such growth, and/or to influence the life processes of plants in a way different from nutrients (e.g. growth regulators). Growth regulators serve, for example, to increase stability in cereals by shortening culm length (culm shorteners or, better, internode shorteners), improve root development of seedlings, reduce plant height by stunting in horticulture, or prevent germination of potatoes. Further examples of crop protection products are herbicides, fungicides and pesticides (e.g. insecticides).

The formulation may also include one or more plant nutrients. What is meant by the term “nutrients” is those inorganic and organic compounds from which plants can extract the elements from which their bodies are formed. These elements themselves are often also referred to as nutrients. These are mostly simple inorganic compounds such as nitrate (NO₃⁻) , phosphate (PO₄³⁻) and potassium (K⁺). In addition to the core elements of organic matter (C, O, H, N and P), K, S, Ca, Mg, Mo, Cu, Zn, Fe, B, Mn, Cl in higher plants, Co, Ni are also vital. Different compounds may be present for the individual nutrients; for example, nitrogen can be supplied as nitrate, ammonium or amino acid.

The at least one material is a material that comes into contact with the formulation in the course of production, finishing, transportation, storage and/or use of the formulation. The at least one material is preferably a material which is in contact with the formulation for a period of time greater than one hour, preferably greater than one day, even more preferably greater than one week, most preferably greater than one month. The period of time may also be more than one year.

The at least one material is preferably a material from which a container for a formulation can be produced and/or a material which is part of a container for a formulation and/or a container itself and/or a material for a lid of a container or a lid of a container and/or a material for a seal of a container and/or a seal for a container.

The at least one material is preferably a plastic or a composite composed of two or more plastics. Materials that are frequently used for contact with a formulation are, for example, polyethylene (PE), polypropylene (PP), polyvinylchloride (PVC), polyethylene terephthalate (PET), polyamide (PA), ethylene-vinyl alcohol copolymer (EVOH), coextrudate of polyethylene and ethylene-vinyl alcohol copolymer (COEX PE/EVOH), coextrudate of polyethylene and polyamide (COEX PE/PA).

The at least one stability feature is a measure of the stability of the system comprising the formulation and the at least one material in contact with the formulation. Stability means the property that at least one of the components of the system, i.e. the formulation or the at least one material, or both components of the system in contact with one another, retain their properties (including their chemical composition) that they had prior to contact. High stability may be manifested, for example, in that the two components of the system retain their properties after contacting or the properties change to an extent of no significance for the intended use of the formulation and/or the at least one material (no quality losses).

Low stability may be manifested, for example, in that at least one property of at least one component of the system after contacting is altered to such a degree that the altered property has an adverse effect on the intended use of the formulation and/or the at least one material.

In the case of low stability, the at least one material will typically be unsuitable as a container or lid or sealant or the like for the formulation. In the case of low stability, the at least one material should not be used as a material in contact with the formulation.

In one embodiment, stability of the system means the resistance of the material to the formulation with which the material is in contact. The formulation may, for example, affect the strength and/or integrity of the material. High stability of the system may therefore mean that the material in contact with the formulation retains its integrity with respect to the formulation and/or with respect to outside moisture and/or with respect to other substances penetrating from the outside (e.g. oxygen). High stability of the system may mean that the material retains its strength in contact with the formulation and does not soften or become brittle and/or retains its shape.

Examples of further stability features are adduced further down in the description.

The at least one stability feature is predicted with the aid of a machine learning model.

A “machine learning model” can be regarded as a computer-implemented data processing architecture. The model is able to receive input data and to supply output data on the basis of said input data and model parameters. The model can learn a relationship between the input data and the output data by means of training. During training, the model parameters can be adjusted so as to supply a desired output for a particular input.

During the training of such a model, the model is presented with training data from which it can learn. The trained machine learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum.

In training, a loss function can be used to control the training process and to assess the prediction quality of the model. The loss function can be chosen such that it assumes low values for a desirable relationship between output data and target data and/or assumes high values in the case of an unwanted relationship between output data and target data. Such a relationship can be, for example, a similarity, a dissimilarity, or another relationship.

A loss function can be used to calculate a loss for a given pair comprising output data and target data. The aim of the training process can consist in altering (adapting) the parameters of the machine learning model so that the loss for all pairs of the training data set is reduced to a (defined, desired) minimum.

A loss function can quantify e.g. the difference between the output data of the model for specific input data and the target data. For example, if the output data and the target data are numbers, the loss function can be the absolute difference between these numbers. In this case, a high absolute value of the loss function can mean that one or more model parameters have to be changed to a great extent.

In the case of output data in the form of vectors, for example, difference metrics between vectors such as the mean squared error, a cosine distance, a norm of the difference vector such as a Euclidean distance, a Chebyshev distance, an Lp norm of a difference vector, a weighted norm, or any other type of difference metric of two vectors can be chosen as the loss function.

In the case of higher-dimensional outputs, such as two-dimensional, three-dimensional or higher-dimensional outputs, an element-by-element difference metric can for example be used. Alternatively or additionally, the output data may be transformed into for example a one-dimensional vector before calculation of a loss value.

In the present case, the machine learning model is trained to predict at least one stability feature for a system comprising a formulation and at least one material in contact with the formulation.

The machine learning model can be trained to predict a single stability feature. Multiple stability features can be predicted using multiple machine learning models, each of which is trained to predict a (single) stability feature.

However, it is also possible that a machine learning model is trained to predict more than one stability feature (e.g. two or three or four or five or six for more than six stability features).

The machine learning model can be trained to predict one or more stability features for a defined material. In order to predict one or more stability features for multiple materials, it is possible to independently train multiple machine learning models each to predict one or more stability features for a defined material. However, it is also possible to train a machine learning model to predict one or more stability features for multiple materials.

The machine learning model is trained with training data. The training data comprise, for a multitude of reference formulations, a recipe and at least one preferably measured stability feature. In this description, by way of distinction of training data and data that are used for prediction, the addition “reference” is used for those formulations from which information is used to train the machine learning model. That formulation for which at least one stability feature is predicted with the aid of the trained machine learning model is also referred to in this description as “test formulation”. The terms “reference formulation” and “test formulation” thus serve merely for distinction between the training phase and the prediction phase. The addition “reference” and the addition “test” otherwise have no kind of (limiting) meaning. Statements that are made in this description for formulations are applicable both to test formulations and to reference formulations. The term “test formulation/reference formulation” is also used in some places in order to make it clear that the respective statement is applicable both to test formulations and to reference formulation.

The expression “multitude of reference formulations” means more than 10, preferably more than 100, reference formulations.

As well as a recipe and at least one preferably measured stability feature, the training data may comprise further data, for example information relating to the material used (for example which material is in contact with the formulation), data for the conditions under which the at least one preferably measured stability feature was ascertained, e.g. measured. The at least one stability feature may, for example, be a property of the material and/or of the formulation after storage under defined conditions. Further training data may, for example, be the duration of storage and/or data relating to the defined conditions (temperature, pressure, strength of a mechanical load and/or the like). The recipe and any further data can be used to generate input data to train the machine learning model; data for the at least one stability feature function as target data in the training of the machine learning model.

The at least one stability feature in the case of training data has preferably been measured, meaning that it is preferably the result of a metrological analysis of each reference formulation and/or the at least one material. The at least one stability feature has preferably been ascertained in a standardized analysis method (for example in a measurement method controlled by a standard (DIN or ISO)). One example of a standard for ascertaining stability features is EN ISO 13274 in its current version.

Typically, in such an analysis method, the at least one material (for example in the form of a test specimen and/or in the form of a consumable article used as intended (for example as a container, lid, seal and/or the like)) is subjected to a first measurement, then contacted with the reference formulation for a defined period of time under defined conditions and then subjected to a second measurement. It is possible that more than two measurements are conducted in order to ascertain the at least one stability feature. It is conceivable that no measurement is conducted prior to the contacting of the at least one material with the reference formulation. It is also conceivable that one or more measurements are conducted on the formulation before and/or after the contacting of the formulation with the material, for example in order to identify one or more substances in the formulation that have been transferred from the material into the formulation.

It is possible that no measured stability feature exists or only some of the stability features are measured for one or more reference formulations. It is possible that one or more stability features of the training data are the result of a calculation.

Examples of stability features are:
swelling of the at least one material in contact with the formulation, for example measured by the method BAM-GGR 004, BAM-GGR 015, DIN EN ISO 13724 with the aid of an analytical balance (for example from Mettler Toledo),
permeation of constituents of the formulation into and/or through the at least one material, for example measured by the method BAM-GGR 015 with the aid of an analytical balance (for example from Mettler Toledo),
tensile impact resistance of the at least one material after contact with the formulation, for example measured by the method BAM-GGR 015 with the aid of a tensile impact hardness tester by the pendulum impact method (e.g. the HIT5P instrument from Zwick Roell GmbH),
melt flow rate of the at least one material after contact with the formulation, for example measured by the method BAM-GGR 015, DIN EN ISO 13724 with the aid of a melt index tester (e.g. the Mflow flow tester from Zwick Roell GmbH),
residual tensile strength of the at least one material after contact with the formulation, for example measured by the method BAM-GGR 004, DIN EN ISO 13724 with the aid of a tensile force tester (e.g. the Z010 universal tester from Zwick Roell GmbH).

BAM-GGR004: https://tes.bam.de/TES/Content/DE/Downloads/ggr-004

BAM-GGR015: https://tes.bam.de/TES/Content/DE/Downloads/ggr-015

As well as the at least one stability feature, the training data include a recipe for each reference formulation.

A recipe comprises information about the constituents of a formulation. A recipe states which constituents are present in a formulation and from which constituents it is formed.

In the case of a crop protection product formulation, customary constituents are: one or more active ingredients (see, for example, https://www.proplanta.de/Pflanzenschutzmittel/Wirkstoffe/), natural oils (e.g. sunflower oil, optionally dewaxed, mineral oils, silicone oils), fatty acids and fatty acid derivatives (e.g.), aliphatic solvents (e.g. butyl lactate, cyclohexanone, dimethyl sulfoxide, ethanol), aromatic solvents (e.g. toluene, Solvesso 100, Solvesso 150, Solvesso 200, Solvarex, Caromax 28 LN, Caromax 20 LN, xylene), surfactants (e.g. ATLOX 3467, Enviomet EM 5665. Enviomet WT 8519, Rhodacal 60-BE, Genapol X-060, Genapol XM 060), waxes (e.g. Joncryl Wax 4), further additives (e.g. dyes such as Laicril P-1530).

The recipe also states the amounts of the constituents. The amounts may be stated, for example, as relative parameters (for example in percent by weight, mole percent, percent by volume, concentration) or as absolute parameters (e.g. mass, moles, volume) in the recipe.

The recipe may include information as to how the formulation is produced; in other words, it may include information, for example, as to the sequence in which the constituents are combined and/or under what conditions they are combined (e.g. temperature, pressure, stirrer speed), how they are combined (e.g. dropwise, with stirring and/or the like) and/or what other steps are undertaken for production of the formulations (e.g. filtering, sedimenting, distilling, heating, cooling and/or the like).

The recipe preferably includes information about the type of (reference) formulation. Examples of types of (reference) formulations are: emulsion concentrate formulations (EC formulations), suspension concentrate formulations (SC formulations), suspoemulsions (SE formulations), oil dispersions (OD formulations), emulsions for seed treatment (ES formulations), oil-in-water emulsion (EW formulations), suspension concentrate for seed treatment (FS formulations, FS: flowable concentrate for seed treatment), technical grade active ingredients (TC formulations, TC: technical concentrate), mixed formulations composed of CS and SC (ZC formulations).

Further examples can be found inter alia at https://www.bvl.bund.de/EN/Tasks/04_Plant_protection_products/01_ppp_tasks/08_ProductChemistr y/01_ppp_coformulants_formulationChemistry/01_ppp_formulation_types/ppp_formulation_types_no de.html.

In a first step of the training method and the prediction method, constituents of the test formulation/reference formulations are ascertained on the basis of the recipe. The constituents are typically specified in the form of a name (e.g. IUPAC name or common name or brand name), a code (e.g. Chemical Abstract Service Registry Number (CAS No.)) and/or a formula (e.g. empirical formula, chemical structural formula).

Preferably at least the main constituents of the respective formulation(s) are ascertained. A main constituent may, for example, be a constituent present in the formulation in a minimum amount (for example at least 1% or at least 5% or at least 10%, where the percentage may be, for example, percent by weight or percent by volume or mole percent).

Concealed behind some constituents of a recipe is not a uniform chemical compound but a mixture of different chemical compounds (e.g. sunflower oil). Preferably, for constituents of a formulation that consist of more than one chemical compound, chemical compounds present in the constituent are ascertained. What is meant by the term “chemical compound” is a pure substance consisting of atoms of two or more chemical elements, where the types of atom-by contrast with mixtures-are in a fixed stoichiometric ratio to one another. Preferably, the chemical compounds of a constituent that are ascertained are at least those that are present in the constituent in a minimum amount (for example at least 1% or at least 5% or at least 10%, where the percentage may be, for example, percent by weight or percent by volume or mole percent). Preferably all the chemical compounds that are respectively present in a constituent are ascertained.

Preferably all the constituents of the respective formulation(s) are ascertained. Which chemical compound(s) are included is preferably ascertained for each constituent. In other words: if this description refers to a “constituent”, this preferably means a chemical compound (and not a generic constituent).

For a multitude of constituents comprising more than one chemical compound, the chemical compounds present can be ascertained from publicly accessible databases and/or from the literature. It is often the case that the manufacturers of constituents give information about the chemical compounds present, for example in the form of a product specification. It is also possible to analyse/commission analysis of constituents for the chemical compounds present.

Some constituents may contain impurities. Impurities are preferably ascertained for constituents. Impurities are preferably ascertained at least for the main constituents of the respective formulation(s). Preferably at least those impurities that occur in a minimum amount in a (main) constituent are ascertained. For a multitude of constituents, the impurities present can be ascertained from publicly accessible databases and/or from the literature. It is often the case that the manufacturers of constituents give information about the impurities present, for example in the form of a product specification. It is also possible to analyse/commission analysis of constituents for the impurities present. Impurities present are preferably taken into account in the form of the respective chemical compound(s).

According to the formulation, the constituents ascertained may be more than one hundred constituents, especially when the chemical compounds of which the constituents and/or impurities are composed are ascertained.

Molecular descriptors are preferably ascertained for preferably all the constituents ascertained (chemical compounds) in the respective formulation(s), especially for those constituents (chemical compounds) for which no measured values of physical and/or chemical properties are available.

What is meant by the term “molecular descriptor” is a representation of a chemical compound that reflects the chemical structure of the chemical compound at the molecular level or from which the chemical structure can be derived at the molecular level.

Examples of molecular descriptors are the IUPAC name (IUPAC: International Union of Pure and Applied Chemistry), SMILES code (SMILES: Simplified Molecular Input Line Entry Specification), InChI code, CML code (CML: Chemical Markup Language), WLN code (WLN: Wiswesser Line Notation), molecular graph.

Molecular descriptors can be ascertained, for example, from publicly accessible databases and/or from the literature using the name of a constituent or using a code for the constituent. It is often the case that the manufacturers of constituents also give molecular descriptors of chemical compounds, for example in the form of a product specification. Molecular descriptors can be created manually by means of suitable programs (e.g. ChemDraw, ChemSketch). Molecular descriptors can typically be interconverted, i.e. an IUPAC name can be used to create a SMILES code, for example, and vice versa.

The molecular descriptors ascertained are preferably those that can be used in computer programs for ascertaining physical and/or chemical properties of chemical compounds on the basis of the structure of the chemical compounds. These are typically SMILES codes and/or molecular graphs. More with regard to computer programs for ascertaining physical and/or chemical properties is given further down in the description.

Molecular descriptors are preferably ascertained at least for the main constituents of a formulation and/or for chemical compounds and/or impurities present at least in a minimum amount in a (main) constituent.

Molecular descriptors are preferably ascertained for all chemical compounds of a formulation including impurities.

For constituents of the test formulation/reference formulations, physical and/or chemical properties are ascertained. Examples of physical and/or chemical properties are:
molecular weight,
partition coefficient, for example for n-octanol/water, or a derived parameter, for example the decadic logarithm of the partition coefficient logD, for example at pH=7 and/or pH=2.3, and/or the logP value,
melting point (for example under standard conditions),
water solubility, for example in the form of the solubility as mass of substance of the compound that can be dissolved in 100 g of water,
proportion of polar/apolar functional groups in one or more molecules present,
pKa/pKb values of one or more molecules present,
charges in the molecule at corresponding pH,
pH of the formulation.

The physical and/or chemical properties may be measured values. The physical and/or chemical properties may be read from one or more databases and/or ascertained from the literature. Examples of databases in which physical and/or chemical properties are stored are PubChem (https://pubchem.ncbi.nlm.nih.gov/), NIST Chemistry WebBook (https://webbook.nist.gov/chemistry/) and CRC Handbook of Chemistry and Physics (https://hbcp.chemnetbase.com/) The physical and/or chemical properties may be ascertained experimentally.

The physical and/or chemical properties may be calculated values. The physical and/or chemical properties can be calculated, for example on the basis of the molecular structure of a constituent. There are numerous methods of calculating physical and/or chemical properties on the basis of the molecular structure. These can be found in the literature under the term QSPR (quantitative structure-property relationship) inter alia. There are also commercially and freely available computer programs for calculation of physical and/or chemical properties of substances based on their molecular structure (e.g.: QSAR-Co: J. Chem. Inf. Model. 2019, 59, 6, 2538-2544; Molgen-QSPR: https://www.researchgate.net/publication/266470632).

A method of calculating the decadic logarithm of the partition coefficient for n-octanol/water is described, for example, in: A. J. Leo: Calculating log P_{oct} from structures, Chem. Rev. 1993, 93, 4, 1281-1306.

A method of calculating the melting point is described, for example, in: H. Modarresi et al.: QSPR Correlation of Melting Point for Drug Compounds Based on Different Sources of Molecular Descriptors, J. Chem. Inf. Model. 2006, 46, 2, 930-936.

A method of calculating the water solubility is described, for example, in: N. Meftahi et al.: Predicting aqueous solubility by QSPR modeling, Journal of Molecular Graphics and Modelling, Volume 106, July 2021, 107901.

Further methods of calculating physical and/or chemical properties can be found in: A. R. Katritzky et al.: QSPR as a means of predicting and understanding chemical and physical properties in terms of structure, Pure and Applied Chemistry 69(2):245-248.

Physical and/or chemical properties of the constituents are preferably calculated on the basis of the respective molecular descriptor for all constituents (chemical compounds) of a formulation for which a molecular descriptor has been ascertained.

In a further step, a numerical representation is created for each formulation.

Many machine learning models require numerical values as input data. The input data that represent a formulation include a respective numerical representation of the respective constituent (the respective chemical compound) for each ascertained constituent (each chemical compound) of the formulation. It is possible that there is a numerical representation for each constituent (each chemical compound) of the formulation. It is possible that there is a numerical representation only for main constituents of the formulation.

A numerical representation of a constituent (a chemical compound) comprises the amount of the constituent (the chemical compound) in the formulation and values for the ascertained physical and/or chemical properties of the constituent (the chemical compound). The numerical representation preferably consists exclusively of the amount of the constituent (the chemical compound) in the formulation and values for the ascertained physical and/or chemical properties of the constituent (the chemical compound). The numerical representation of a constituent (a chemical compound) preferably does not include any category variables. The amount of the constituent (the chemical compound) and the values for the ascertained physical and/or chemical properties of the constituent (the chemical compound) may take the form of a vector for example.

In other words: prediction of the at least one stability feature is based, in a preferred embodiment, on numerical representations of the constituents (chemical compounds) of a formulation that comprise merely information as to the amount of constituents (chemical compounds) and the physical and/or chemical properties thereof. There is no requirement for further information, for example information as to the type of constituents (that can be encoded with category variables) or as to the chemical structures of the chemical compounds of the constituents. This further information is implicitly included in the physical and/or chemical properties. In a preferred embodiment, all physical and/or chemical properties are calculated on the basis of the molecular descriptors (that represent the chemical structure). It is thus possible to represent a formulation solely via physical and/or chemical properties of its constituents and the amounts of the constituents. Such a representation is sufficient to be able to predict a stability feature for a system comprising the formulation and a material in contact with the formulation.

A numerical representation of a formulation may consist exclusively of the numerical representations of its (main) constituents. It may, for example, be a matrix in which each line or each column is a numerical representation of a constituent.

The numerical representation of each formulation preferably comprises, aside from numerical representations of constituents of the representation, preferably only one or more category variable(s) that specify/specifies the type of formulation. For example, an SC formulation may be represented by a 1, an SE formulation by a 2, and other formulations by other numbers. The type of formulation may also be represented by a 1-from-n code (one-hot-encoding).

In the training of the machine learning model, input data that represent a reference formulation are input into the machine learning model. The machine learning model may be configured to subject the numerical representations of the constituents independently to a feature extraction in which an optionally compressed numerical representation of the constituent is created from each numerical representation of a constituent. Each numerical representation of a constituent may be fed, for example, to an artificial neural network configured to create an optionally compressed numerical representation based on the numerical representation fed in and on the basis of trainable (learnable) model parameters. The artificial neural network may, for example, be a convolutional neural network (CNN).

The numerical representation of a formulation in the feature space contains information of relevance for the prediction of the at least one stability feature. However, it may also contain other irrelevant or redundant information. A “compressed numerical representation” is a representation that contains or emphasizes only the relevant information or in which irrelevant and/or redundant information is suppressed and/or reduced.

A compressed numerical representation can be created, for example, by an encoder of an autoencoder.

An autoencoder is generally used to learn efficient data coding in an unsupervised learning method. In general, the purpose of an autoencoder is to learn a representation for a dataset, typically for reduction of dimensionality, in that the machine learning model is trained to identify the relevant data and ignore noise.

The encoder compresses the input data into a representation in latent space, and the decoder attempts to restore the input data from this representation in latent space. The representation in latent space is the compressed numerical representation of the input data.

A main feature of an autoencoder is an information bottleneck between the encoder and decoder. This bottleneck, a continuous vector of fixed length, causes the machine learning model to learn a compressed representation that covers the most statistically relevant information in the data.

Further details of autoencoders and the creation of compressed representation can be found in various publications (see, for example, D. Bank et al.: Autoencoders, arXiv:2003.05991).

The numerical representations of formulations may be of different size for different formulations (i.e. have different numbers of dimensions), merely because they can have a different number of constituents.

Therefore, in a first step, for each ascertained constituent of a formulation, an optionally compressed numerical representation of the constituent can be created on the basis of the numerical representation of the constituent. In a second step, the optionally compressed numerical representations of all the ascertained constituents of a formulation can be summarized by means of a pooling operation in a numerical representation of the formulation. The result may be a representation (a vector) that has the same size (the same dimension) for all formulations.

It is possible to use an attention mechanism in order to combine numerical representations of constituents. In machine learning, attention is a technique that emulates cognitive attention in man. The effect is that some parts of the input data are amplified and other parts are attenuated-the idea is that the machine learning model should pay more attention to an important part of the data than to a less important part. Which parts are important and which parts are less important is learned by the model in the training phase.

Although it is possible for model architectures based on convolutional neural networks to describe any functions on mathematical sets (see, for example, M. Zaheer et al.: Deep Sets, arXiv:1703.06114v3), such architectures can only implicitly model individual interactions between particular formulation constituents from the same recipe. Neural networks having an attention mechanism, by contrast, are capable of explicitly modelling interactions between formulation constituents of the same recipe. Attention functions, in quite general terms, combine a query vector with pairs of (key, value) vectors to give an output vector. The output vector in turn is calculated from the weighted sum of the value vectors, where the weights are predicted by the network architecture on the basis of the query and key vectors. By providing physical/chemical properties of formulation constituents from a recipe as query, key and value vectors, it is possible to directly process interactions between formulation constituents of the same recipe. It has been found that the attention mechanism can improve the prediction accuracy of the trained models compared to convolutional neural networks.

It is possible to use a transformer network to create a numerical representation of a formulation. Such a transformer network can take account of pairs of interactions between the elements of the input. The transformer network can process sequential inputs and give different weight to the contributions of the individual parts of the input data. The use of multiple self-attention units makes the transformer network capable of processing relevant interactions between two or more constituents of the formulation. Details of transformer networks can be found, for example, in: J. Lee et al.: A Framework for Attention-based Permutation-Invariant Neural Networks, 2019, arXiv:1810.00825v3; A. Vaswani et al.: Attention is all you need, arXiv:1706.03762v5.

Further details of attention mechanisms are described in scientific articles and patents/patent applications (see, for example: A. Vaswani et al.: Attention Is All You Need, arXiv:1706.03762v5, EP3166049A1, US20170262996, WO2020/222985).

Finally, the recipe of a formulation can be encoded in a single vector via a global pooling operation (see, for example: J. Lee et al.: Set Transformer: A Framework for Attention-based Permutation-Invariant Neural Networks, arXiv:1810.00825v3). An example of a global pooling operation is the summation of the representation (or embedding preprocessed by the attention mechanism) of the individual constituents. A more flexible extension of summation is attention pooling. Inspired by the attention mechanism, a single learnable vector is provided as a query vector, with which the representations (or embeddings) of the formulation constituents as key and value vectors can be used to create a single output vector. Here too, representations of formulation constituents are processed in a weighted sum, except that the weights are created from the representations (or embeddings) of the formulation constituents.

The pooling operation gives rise to a permutation-invariant representation of a formulation. This means that the sequence in which constituents are listed is immaterial. In other words, for a formulation comprising constituents A, B and C, the same representation is always created, irrespective of whether the constituents of the formulation are listed in a representation of the formulation in the sequence of A, B, C or A, C, B or B, A, C or B, C, A or C, A, B or C, B, A.

The (permutation-invariant) representation of the formulation may be sent to a regression model configured to calculate at least one stability feature on the basis of the (permutation-invariant) representation of the formulation and on the basis of trainable (learnable) model parameters.

The regression model may, for example, be an artificial neural network or a random forest model or the like. Various examples of regression models can be found in the literature (see, for example: H. Saleh, J. A. Layous: Machine Learning—Regression, 2022, DOI: 10.13140/RG.2.2.35768.67842). In one embodiment, the regression model is an artificial neural network which is trained together with the upstream networks to pool the representations of the formulation constituents. With regard to the architecture, such a regression network may be a “fully connected network” (also referred to as a “multilayer perceptron”). In this architecture, individual neurons (with an input and an output) are organized in layers, such that the totality of the outputs of one layer constitutes the input of each neuron of the subsequent layer.

The at least one predicted stability feature may be compared with the at least one (preferably measured) stability feature of the training data. The variances between the at least one predicted stability feature and the stability feature of the training data can be quantified with the aid of a loss function. Examples of loss functions are root mean square deviation (RMSD), mean absolute deviation (MAD), log-cosh distances, cosine distances, Huber loss, etc.

The model parameters can be modified, for example, in a gradient method or in another optimization method in order to reduce the variances to a (defined) minimum.

The training is continued with further training sets each comprising a numerical representation of a reference formulation and at least one (preferably measured) stability feature.

Once the machine learning model reaches a desired prediction accuracy, the training can be ended. The trained machine learning model can be stored in a data storage medium and/or transmitted to a separate computer system.

The model can be validated by dividing the training data into two datasets. The first dataset can be used for training; the second dataset can be used for validation.

Once the machine learning model has been trained, it can be used to predict at least one stability feature of a new system comprising a new formulation (test formulation) and at least one material in contact with the new formulation (test formulation). What is meant here by the word “new” is that a dataset that describes the formulation has not already been used in training.

The prediction proceeds analogously to the training. This means that the steps that have been conducted in relation to the reference formulations have also been conducted for the new formulation (test formulation):

In a first step, a recipe of the test formulation is received.

In a second step, constituents of the test formulation are ascertained on the basis of the recipe.

In a third step, physical and/or chemical properties of the constituents of the test formulation are ascertained.

In a fourth step, a numerical representation of the test formulation is created. The numerical representation comprises a numerical representation of the ascertained constituent for each constituent ascertained. Each numerical representation of a constituent ascertained comprises a value for the amount of the constituent in the test formulation and values for the ascertained physical and/or chemical properties of the constituent.

In a fifth step, the numerical representation of the test formulation is input into the trained machine learning model.

In a sixth step, at least one predicted stability feature is received from the trained machine learning model.

In a seventh step, the at least one predicted stability feature is output (for example displayed on a screen and/or printed out on a printer) and/or stored in a data storage medium and/or transmitted to a separate computer system.

In a further step, the prediction can be experimentally verified. It is possible that there is verification by means of a measurement as to whether the at least one predicted stability feature has been correctly predicted.

It is possible that not every predicted stability criterion is experimentally verified. It is possible that there is (initially) verification as to whether the at least one predicted stability feature meets predefined requirements. It is possible that the at least one predicted stability feature is compared with a reference value or with multiple reference values. Such a reference value may, for example, be a minimum value that has to be attained for the at least one material for which the prediction has been made to be useful at all as material for a packaging, a container, a seal and/or the like for the test formulation. If a predicted stability feature is smaller than the minimum value, for example, it may be the case that the at least one material is not a useful packaging material, container material, seal material and/or the like for the test formulation. A different material has to be found for the test formulation. If a predicted stability feature is greater than or equal to the minimum value, the material is a candidate for consideration. It is conceivable that the predicted stability feature is verified by an experimental analysis in a laboratory. In other words: if the at least one predicted stability feature meets predefined requirements, an analysis of the at least one material in a laboratory can be initiated, for example by automated creation and transmission of a message to the laboratory and/or automated ordering of one or more constituents of the test formulation and/or automated ordering of the test formulation and/or automated ordering of the at least one material.

The invention is more particularly elucidated hereinafter with reference to drawings, without wishing to restrict the invention to the features and combination of features that are shown in the drawings.

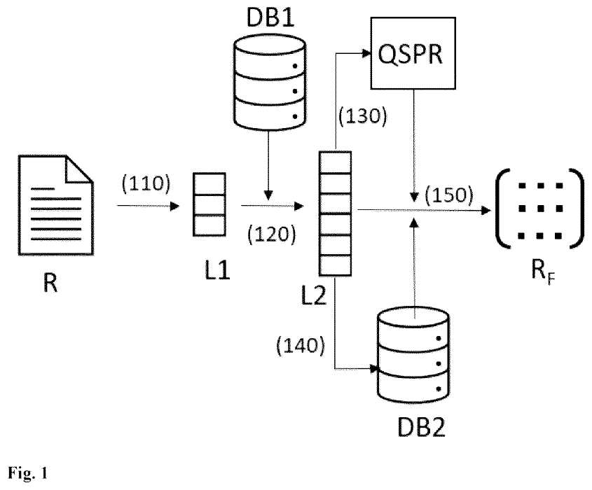

FIG. 1 shows, in a schematic and illustrative manner, the creation of a numerical representation of a test formulation/reference formulation on the basis of a recipe.

The starting point is a recipe R of the respective formulation. In a first step (110), constituents of the formulation are ascertained on the basis of the recipe R, and a first list L1 of constituents ascertained is created. In a second step (120), for constituents in the first list L1 that comprise more than one chemical compound and/or for constituents comprising impurities, chemical compounds of the constituents or impurities are ascertained. The information relating to chemical compounds and/or impurities present in constituents can be obtained from one or more databases DB1. The chemical compounds and/or impurities ascertained are included in a first list L1 of constituents to obtain a second list L2 of constituents.

In a third step (130), for one or more constituents of the formulation, physical and/or chemical properties are calculated on the basis of one or more molecular descriptors of the constituent(s). The calculation is effected with the aid of one or more QSPR models which relate structural properties to physical and/or chemical properties. In addition to or instead of the calculation of physical and/or chemical properties, in a fourth step (140), for one or more constituents of the formulation, physical and/or chemical properties are read from one or more databases DB2.

In a fifth step (150), a numerical representation RF of the formulation is created. The numerical representation R_{F} encompasses, for each constituent ascertained in the second list L2, a value for the amount of the constituent in the formulation and values for the ascertained (calculated and/or read) physical and/or chemical properties.

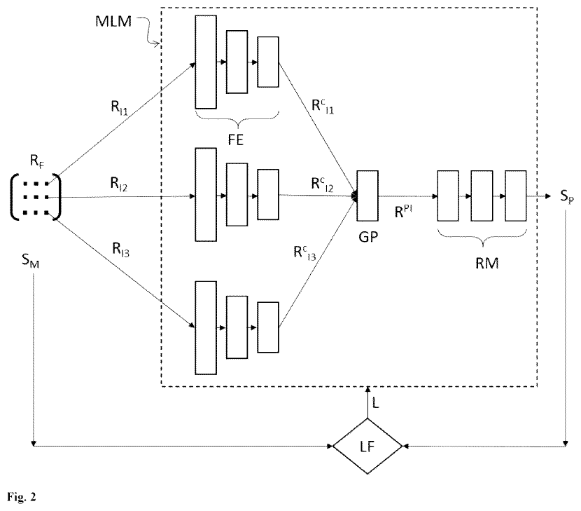

FIG. 2 shows, in a schematic and illustrative manner, an embodiment for the training of the machine learning model. The training is effected with the aid of training data. The training data are obtained on the basis of information relating to a multitude of reference formulations and preferably measured stability features. The example shown in FIG. 2 shows only one training dataset TD comprising a numerical representation R_{F} of a reference formulation and a stability feature S_{M}. The numerical representation R_{F} of the reference formulation can be created as described in relation to FIG. 1. The numerical representation R_{F} comprises the numerical representations R_{I1}, R_{I2} and R_{I3} of three constituents of the reference formulation. However, the number of constituents is not limited to three. Each reference formulation comprises at least one constituent, preferably at least ten constituents and in some cases even more than 50 constituents (there is no upper limit to the number of constituents). The stability feature S_{M} is a measure of the stability of a system comprising the reference formulation and at least one material in contact with the reference formulation. The stability feature S_{M} is preferably the result of a metrological analysis.

The numerical representation R_{F} of the reference formulation is fed as input data to the machine learning model MLM.

In the architecture of the machine learning model (convolutional neural network) shown in FIG. 2, the numerical representations R_{I1}, R_{I2} and R_{I3} of the constituents of the reference formulation are independently subjected to a feature extraction FE. In the feature extraction FE, the numerical representation R_{I1} is used to obtain a numerical representation R^{C}₁₁ that may be compressed (i.e. comprises fewer dimensions than the input representation), but is expanded in a preferred implementation (i.e. comprises more dimensions than the input representation). The numerical representation R_{I2} is likewise used to create a numerical representation R^{C}_{I2}, and the numerical representation R_{I3} to create a numerical representation R^{C}_{I3}. FIG. 2 shows the unit for feature extraction FE in triplicate, in order to process the three numerical representations of the three constituents of the reference representation. However, this does not mean that the machine learning model has one unit each for feature extraction per constituent of a formulation. This would be impractical since different formulations typically have a different number of constituents. It is therefore possible that there is only one unit for feature extraction, which successively processes the numerical representations of the constituents of a formulation.

The (optionally compressed) numerical representations R^{C}_{I1}, R^{C}_{I2} and R^{C}_{I3} are combined by a pooling operation GP to give a preferably permutation-invariant representation R^{PI} of the reference formulation. This pooling operation may, in a simple implementation of the architecture, be a standard reduction operation applied to the processed representations R^{C}_{I1}, R^{C}_{I2} and R^{C}_{I3} (for example the sum total, the average, the minimum in terms of elements, the maximum in terms of elements, etc.). In a preferred implementation of the described architecture, the global pooling operation consists of a multi-attention pooling operation. The global pooling operation is not limited to the options mentioned and may be replaced by other global pooling operations.

The preferably permutation-invariant representation R^{PI} of the reference formulation is sent to a regression model RM which, on the basis of the preferably permutation-invariant representation R^{PI}, ascertains (predicts) a stability feature S_{P}. The predicted stability feature S_{P} can be compared with the stability feature S_{M} of the training dataset. A loss function LF is used in order to quantify the variances between the predicted stability feature S_{P} and the stability feature S_{M} of the training dataset. The ascertained loss L can be used to modify model parameters with regard to reduction of the loss L. Model parameters may lie in the unit for feature extraction FE, in the pooling operation and in the regression model.

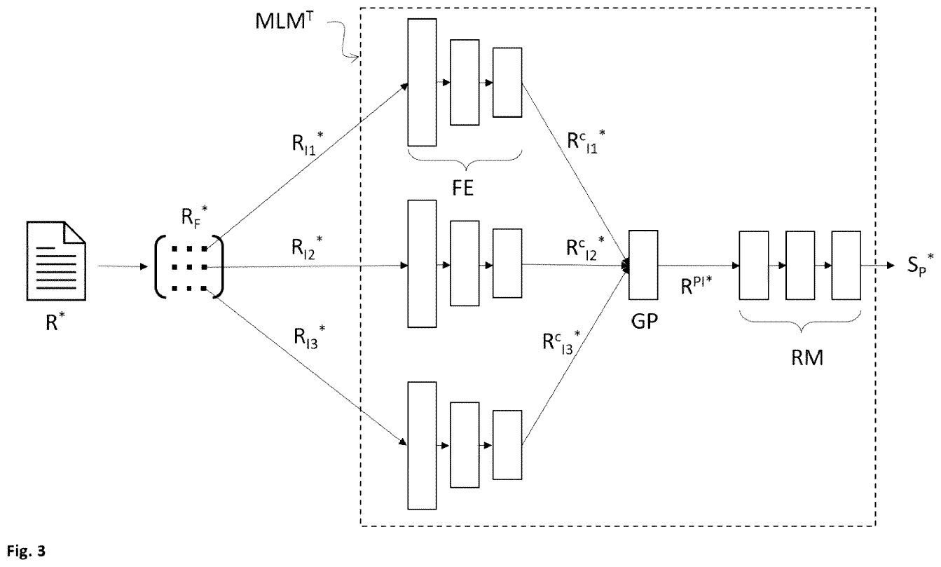

FIG. 3 shows, in schematic form and by way of example, the use of the trained machine learning model for prediction. In the present example, a numerical representation R_{F}* of a test formulation is input into the trained machine learning model MLM^{T}. The numerical representation R_{F}* of the test formulation may be obtained from a recipe R* of the test formulation as described in relation to FIG. 1. The numerical representation R_{F}* of the test formulation comprises, as in the example of FIG. 2, three numerical representations R_{I1}*, R_{I2}* and R_{I3}* of three constituents of the test formulation. These numerical representations R_{I1}*, R_{I2}* and R_{I3}*, analogously to the example of FIG. 2, are used to create three optionally compressed numerical representations R^{C}_{I1}*, R^{C}_{I2}* and R^{C}_{I3}* by feature extraction FE. The optionally compressed numerical representations R^{C}_{I1}*, R^{C}_{I2}* and R^{C}_{I3}* are combined by the pooling operation GP to give a preferably permutation-invariant representation R^{PI}* of the test formulation. The preferably permutation-invariant representation R^{PI}* of the test formulation is sent to the regression model RM, which ascertains (predicts) a stability feature S_{P}* for the test formulation.

FIG. 4 shows, in an illustrative and schematic manner, a computer system of the invention.

A “computer system” is an electronic data processing system that processes data by means of programmable calculation rules. Such a system typically comprises a “computer”, which is the unit that comprises a processor for carrying out logic operations, and peripherals.

In computer technology, “peripherals” denotes all devices that are connected to the computer and are used to control the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 4 comprises an input unit (10), a control and computation unit (20) and an output unit (30).

The control and computation unit (20) is used for control of the computer system (1), for coordination of the data flows between the units of the computer system (1), and for the performance of calculations.

The control and computation unit (20) is configured to
provide the trained machine learning model,
cause the input unit (10) to receive a recipe of a test formulation,
ascertain constituents of the test formulation on the basis of the recipe,
ascertain physical and/or chemical properties of the ascertained constituents,
create a numerical representation of the test formulation, where the numerical representation of the test formulation comprises a numerical representation of the ascertained constituent for each ascertained constituent, where the numerical representation of each ascertained constituent comprises a value for the amount of the constituent in the test formulation and values for the ascertained physical and/or chemical properties of the constituent,
input the numerical representation of the test formulation into the trained machine learning model,
receive at least one predicted stability feature from the trained machine learning model,
cause the output unit (30) to output the at least one predicted stability feature and/or to store and/or to transmit it to a separate computer system.

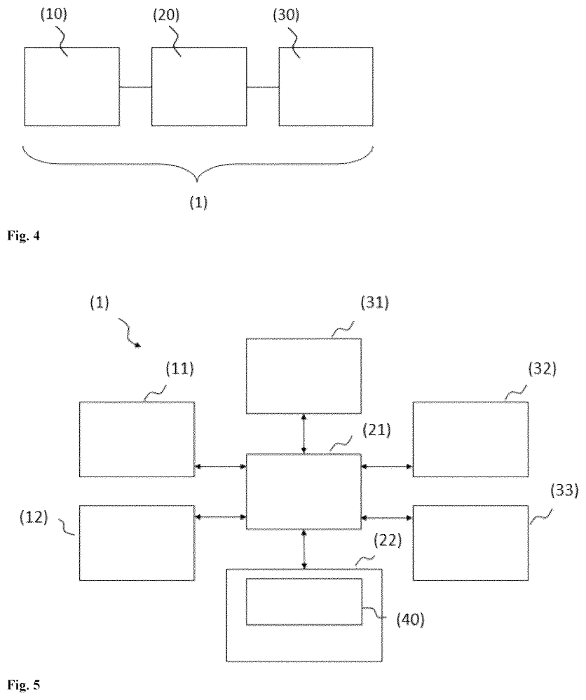

FIG. 5 shows by way of example and in schematic form a further embodiment of the computer system according to the invention.

The computer system (1) comprises a processing unit (21) connected to a memory (22). The processing unit (21) and the memory (22) form a control and computation unit, as shown in FIG. 4.

The processing unit (21) may comprise one or more processors alone or in combination with one or more memories. The processing unit (21) may be standard computer hardware capable of processing information such as digital images, computer programs, and/or other digital information. The processing unit (21) typically consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be standard computer hardware capable of storing information, data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disk, a magnetic tape or a combination of the aforementioned. Optical disks may include compact disks with read-only memory (CD-ROM), compact disks with read/write function (CD-R/W), DVDs, Blu-ray disks, and the like.

The processing unit (21) may be connected not only to the memory (22) but also to one or more interfaces (11, 12, 31, 32, 33) in order to display, transmit and/or receive information. The interfaces may comprise one or more communication interfaces (32, 33) and/or one or more user interfaces (11, 12, 31). The one or more communication interfaces may be configured to send and/or receive information, for example to and/or from other computer systems, networks, data storage media or the like. The one or more communication interfaces may be configured to transmit and/or receive information via physical (wired) and/or wireless communication connections. The one or more communication interfaces may comprise one or more interfaces for connection to a network, for example using technologies such as cellphone, Wi-Fi, satellite, cable, DSL, optical fiber, and/or the like. In some examples, the one or more communication interfaces may comprise one or more close-range communication interfaces configured to connect devices with short-range communication technologies such as NFC, RFID, Bluetooth, Bluetooth LE, ZigBee, infrared (e.g. IrDA) or the like.

The user interfaces may comprise a display (31). A display (31) may be configured to display information to a user. Suitable examples thereof are a liquid-crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This may include barcodes, radiofrequency identification (RFID), magnetic strips, optical character recognition (OCR), integrated circuit cards (ICC) and the like. The user interfaces may also include one or more interfaces for communication with peripherals such as printers and the like.

One or more computer programs (40) may be stored in the memory (22) and executed by the processing unit (21), which is thereby programmed to fulfill the functions described in this description. The retrieving, loading, and execution of instructions in the computer program (40) may take place sequentially, such that an instruction is respectively retrieved, loaded and executed. However, the retrieving, loading and/or execution may also take place in parallel.

The machine learning model of the invention may also be stored in the memory (22).

The computer system of the invention may be executed as a laptop, notebook, netbook and/or tablet PC.

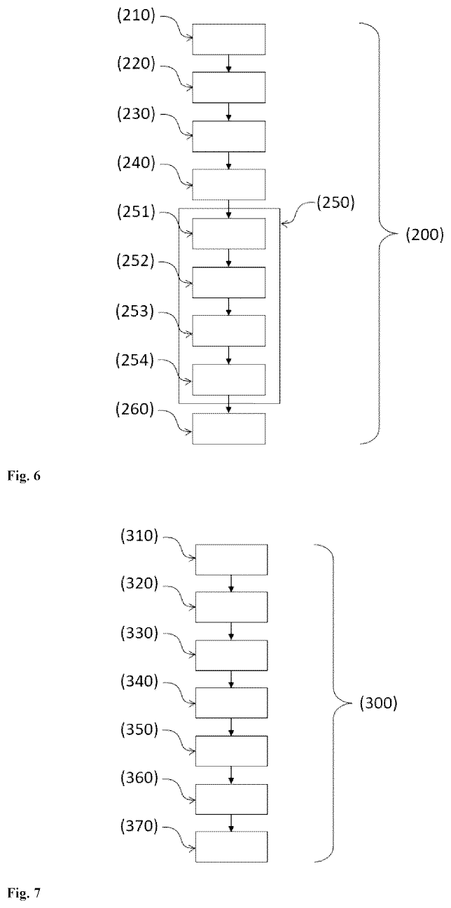

FIG. 6 shows a schematic, in the form of a flow chart, of one embodiment of the method of training the machine learning model. The training method (200) comprises the steps of:
(210) providing and/or receiving information about a multitude of reference formulations, where the information for each reference formulation comprises a recipe and at least one preferably measured stability feature, where the stability feature is a measure of the stability of the system comprising the reference formulation and at least one material in contact with the reference formulation,
(220) ascertaining constituents of the reference formulation on the basis of the recipe,
(230) ascertaining physical and/or chemical properties of the ascertained constituents of each reference formulation,
(240) creating a numerical representation for each reference formulation, where the numerical representation of the reference formulation comprises a numerical representation of the ascertained constituent for each ascertained constituent, where the numerical representation of each ascertained constituent comprises a value for the amount of the constituent in the reference formulation and values for the ascertained physical and/or chemical properties of the constituent,
(250) training the machine learning model, where the training comprises the following steps for each reference formulation:
   (251) inputting the numerical representation of the reference formulation into a machine learning model, where the machine learning model is configured to predict at least one stability feature based on the numerical representation of the reference formulation and model parameters,
   (252) receiving the at least one predicted stability feature from the machine learning model,
   (253) quantifying a variance between the at least one preferably measured stability feature and the at least one predicted stability feature,
   (254) modifying the model parameters with regard to reducing the variance,
(260) storing and/or outputting the trained machine learning model and/or transmitting the trained machine learning model to a separate computer system and/or using the trained machine learning model to predict at least one stability feature of a new system comprising a formulation and at least one material in contact with the formulation.

FIG. 7 shows, in schematic form, in the form of a flow chart, an embodiment of the method of predicting at least one stability feature of a system comprising a test formulation and at least one material in contact with the test formulation by means of the trained machine learning model. The prediction method (300) comprises the steps of:
(310) receiving a recipe of the test formulation,
(320) ascertaining constituents of the test formulation on the basis of the recipe,
(330) ascertaining physical and/or chemical properties of the ascertained constituents,
(340) creating a numerical representation of the test formulation, where the numerical representation of the test formulation comprises a numerical representation of the ascertained constituent for each ascertained constituent, where the numerical representation of each ascertained constituent comprises a value for the amount of the constituent in the test formulation and values for the ascertained physical and/or chemical properties of the constituent,
(350) inputting the numerical representation of the test formulation the trained machine learning model,
(360) receiving at least one predicted stability feature from the trained machine learning model,
(370) outputting and/or storing the at least one predicted stability feature and/or transmitting the at least one predicted stability feature to a separate computer system.

### EXAMPLE

There follows a description of an example for execution of the present invention. The machine learning model used was an artificial neural network that processes physical/chemical representations of individual formulation constituents (based on logD values measured at neutral and acidic pH, water solubility, melting point and molecular mass in combination with information relating to formulation type) in an attention mechanism for material-specific embedding, pools these by means of attention pooling, and then predicts the desired stability features using a fully connected neural network.

The machine learning model was trained on the basis of recipes of different formulations of crop protection products. The machine learning model was trained to predict the following stability features based on a numerical representation of the respective recipe.

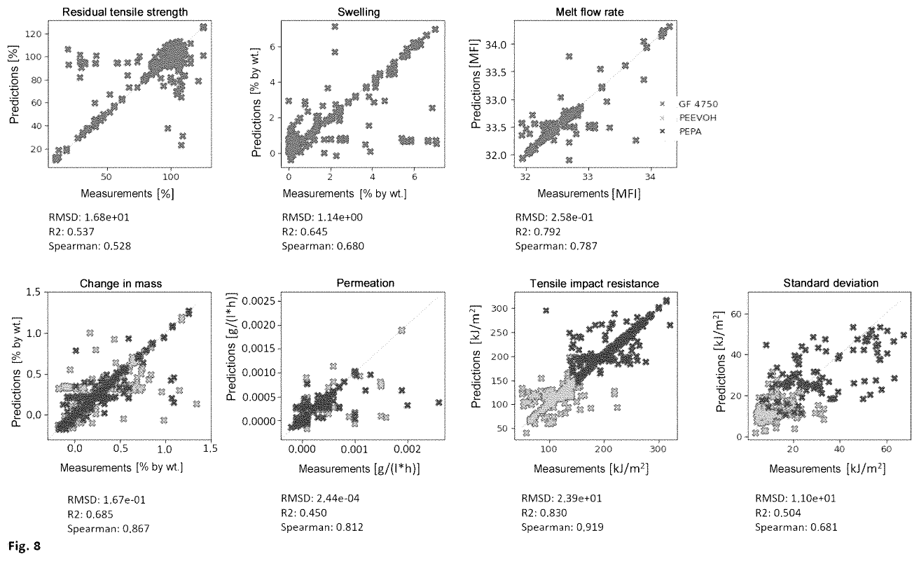

FIG. 8 shows a comparison of measured and predicted stability features. For three different packaging materials (GF 4750: polyethylene, PEEVOH: COEX PE/EVOH, PEPA: COEX PE/PA), a total of seven different stability features were predicted (residual tensile strength, change in mass, permeation, tensile impact resistance, standard deviation of the measurement of tensile impact resistance).

The actual measurements and predictions are close to the diagonals drawn and hence show good agreement.

On the basis of these predicted stability features, it is then possible to estimate suitabilities of the materials as packaging materials for the respective formulation. For example, tensile strength should be 100%. However, this stability feature is below 50% for various materials. Such low values should be avoided.

For particular stability features (e.g. tensile impact resistance), what is important is not just the absolute value but also the scatter of the measurements (“standard deviation”), in order to be able to make a statement with regard to suitability. A prediction of scatter is likewise possible by the machine learning model.

The corresponding predicted stability features can be used to conduct tests for verification of the predictions in a prioritized manner. For this purpose, a limit or criterion for assessment of a predicted stability feature can be defined on the basis of experience and/or on the basis of stability features of known materials. It is possible to conduct measurements with those materials for which a predicted stability criterion is above the defined limit and/or to conduct measurements with those materials for which the best predicted results have been achieved. Materials for which a predicted stability feature is below the defined limit do not require performance of practical storage and/or stability tests since they appear to be unsuitable as container for the respective formulation.

1. A computer-implemented method comprising:
providing a trained machine learning model (MLM^{T}),
wherein the trained machine learning model (MLM^{T}) has been configured and trained with the aid of training data to predict at least one stability feature (S_{P}, S_{P}*) for a system comprising a formulation and at least one material in contact with the formulation based on a numerical representation (R_{F}, R_{F}*) of the formulation,
wherein the training data for each reference formulation of a multitude of reference formulations comprise (i) a numerical representation (R_{F}) of the reference formulation and (ii) at least one measured stability feature (S_{M}), where the at least one measured stability feature (S_{M}) is a measure of the stability of a system comprising the reference formulation and at least one material in contact with the reference formulation,
wherein the training of the machine learning model (MLM) for each reference formulation comprises:
inputting the numerical representation (R_{F}) of the reference formulation into the machine learning model (MLM);
receiving at least one predicted stability feature (S_{P}) from the machine learning model (MLM);
quantifying a variance between the at least one measured stability feature (S_{M}) and the at least one predicted stability feature (S_{P}); and
reducing the variance by modifying the model parameters;
receiving a recipe (R*) of a test formulation;
ascertaining constituents of the test formulation on the basis of the recipe (R*);
ascertaining physical and/or chemical properties of the ascertained constituents;
creating a numerical representation (R_{F} ) of the test formulation, where the numerical representation (R_{F}*) of the test formulation comprises a numerical representation (R_{I1}*, R_{I2}*, R_{I3}*) of the ascertained constituent for each ascertained constituent, where the numerical representation (R_{I1}*, R_{I2}*, R_{I3}*) of each ascertained constituent comprises a value for the amount of the constituent in the test formulation and values for the ascertained physical and/or chemical properties of the constituent;
inputting the numerical representation (R_{F}*) of the test formulation into the trained machine learning model (MLM^{T});
receiving at least one predicted stability feature (S_{P}*) from the trained machine learning model (MLM^{T}); and
outputting and/or storing the at least one predicted stability feature (S_{P}*) and/or transmitting the at least one predicted stability feature (S_{P}*) to a separate computer system.

2. The method as claimed in claim 1, wherein the training of the machine learning model (MLM) further comprises:
providing and/or receiving information about the multitude of reference formulations, where the information for each reference formulation comprises (i) a recipe (R) and (ii) the at least one measured stability feature (S_{M});
ascertaining constituents of the reference formulation based on the recipe (R) of the reference formulation;
ascertaining physical and/or chemical properties of the ascertained constituents of each reference formulation; and
creating a numerical representation (R_{F}) for each reference formulation, where the numerical representation (R_{F}*) of the reference formulation comprises a numerical representation (R_{I1}, R_{I2}, R_{I3}) of the ascertained constituent for each ascertained constituent, where the numerical representation (R_{I1}, R_{I2}, R_{I3}) of each ascertained constituent comprises a value for the amount of the constituent in the reference formulation and values for the ascertained physical and/or chemical properties of the constituent.

3. The method as claimed in claim 1, wherein the test formulation and each reference formulation is a formulation of a crop protection product.

4. The method as claimed in claim 1, wherein the at least one material is a material of a container for a formulation, a material of a lid of a container for a formulation and/or a material of a seal of a container for a formulation.

5. The method as claimed in claim 1, wherein the at least one material is a plastic or a composite composed of two or more plastics.

6. The method as claimed in claim 1, wherein the at least one material for all reference formulations and the test formulation is the same material or two or more of the same materials.

7. The method as claimed in claim 1, wherein the at least one measured stability feature (S_{M}) and the at least one predicted stability feature (S_{P}, S_{P}*) comprises one or more of the following stability features:
swelling of the at least one material in contact with the reference formulation/test formulation,
permeation of constituents of the reference formulation/test formulation into and/or through the at least one material,
tensile impact resistance of the at least one material after contact with the reference formulation/test formulation,
melt flow rate of the at least one material after contact with the reference formulation/test formulation, and
residual tensile strength of the at least one material after contact with the reference formulation/test formulation.

8. The method as claimed in claim 1, wherein the recipe (R*, R) of the test formulation and/or reference formulation includes information about the type of test formulation and/or reference formulation.

9. The method as claimed in claim 1, wherein the ascertaining of constituents of the test formulation and/or reference formulation on the basis of the recipe (R*, R) comprises: ascertaining impurities of constituents.

10. The method as claimed in claim 1, wherein the ascertaining of constituents of the test formulation and/or reference formulation on the basis of the recipe (R*, R) comprises: for constituents of the test formulation and/or reference formulation consisting of more than one chemical compound, ascertaining chemical compounds present in the constituent.

11. The method as claimed in claim 1, further comprising:
ascertaining all chemical compounds of all constituents of the test formulation/reference formulations having a minimum content; and
ascertaining a molecular descriptor for each of the ascertained chemical compounds,
wherein the ascertaining of physical and/or chemical properties of the ascertained constituents comprises:
calculating physical and/or chemical properties of the ascertained chemical compounds on the basis of the molecular descriptors.

12. The method as claimed in claim 1, wherein the ascertained physical and/or chemical properties include one or more of the following properties:
molecular weight,
partition coefficient for n-octanol/water or a parameter derived therefrom,
melting point, and
water solubility.

13. The method as claimed in claim 1, wherein the machine learning model (MLM, MLM^{T}) is configured, for each numerical representation (R_{I1}, R_{I2}, R_{I3}, R_{I1}*, R_{I2}*, R_{I3}*) of each ascertained constituent, to create an optionally compressed numerical representation (R^{C}_{I1}, R^{C}_{I2}, R^{C}_{I3}, R^{C}_{I1}*, R^{C}_{I2}*, R^{C}_{I3}*) and to combine the optionally compressed numerical representations (R^{C}_{I1}, R^{C}_{I2}, R^{C}_{I3}, R^{C}_{I1}*, R^{C}_{I2}*, R^{C}_{I3}*) of the constituents to form a permutation-invariant representation (R^{PI}, R^{PI}*) of the respective reference formulation/test formulation.

14. A computer system comprising:
an input unit;
a control and computation unit; and
an output unit;
where the control and computation unit is configured to:
provide a trained machine learning model (MLM^{T}),
wherein the trained machine learning model (MLM^{T}) has been configured and trained with the aid of training data to predict at least one stability feature (S_{P}, S_{P}*) for a system comprising a formulation and at least one material in contact with the formulation based on a numerical representation (R_{F}, R_{F}*) of the formulation,
wherein the training data for each reference formulation of a multitude of reference formulations comprise (i) a numerical representation (R_{F}) of the reference formulation and (ii) at least one measured stability feature (S_{M}), where the at least one measured stability feature (S_{M}) is a measure of the stability of a system comprising the reference formulation and at least one material in contact with the reference formulation,
wherein the training of the machine learning model (MLM) for each reference formulation comprises:
inputting the numerical representation (R_{F}) of the reference formulation into the machine learning model (MLM);
receiving at least one predicted stability feature (S_{P}) from the machine learning model (MLM);
quantifying a variance between the at least one measured stability feature (S_{M}) and the at least one predicted stability feature (S_{P}); and
reducing the variance by modifying the model parameters ;
cause the input unit to receive a recipe (R*) of a test formulation;
ascertain constituents of the test formulation on the basis of the recipe (R*);
ascertain physical and/or chemical properties of the ascertained constituents;
create a numerical representation (R_{F}*) of the test formulation, where the numerical representation (R_{F}*) of the test formulation comprises a numerical representation (R_{I1}*, R_{I2}*, R_{I3}*) of the ascertained constituent for each ascertained constituent, where the numerical representation (R_{I1}*, R_{I2}*, R_{I3}*) of each ascertained constituent comprises a value for the amount of the constituent in the test formulation and values for the ascertained physical and/or chemical properties of the constituent;
input the numerical representation (R_{F}*) of the test formulation into the trained machine learning model (MLM^{T});
receive at least one predicted stability feature (S_{P}*) from the trained machine learning model (MLM^{T}); and
cause the output unit to output the at least one predicted stability feature (S_{P}*) and/or to store and/or to transmit it to a separate computer system.

15. A computer-readable storage medium comprising commands which, on execution by a computer system, cause the latter to execute the following steps:
providing a trained machine learning model (MLM^{T}),
wherein the trained machine learning model (MLM^{T}) has been configured and trained with the aid of training data to predict at least one stability feature (S_{P}, S_{P}*) for a system comprising a formulation and at least one material in contact with the formulation based on a numerical representation (R_{F}, R_{F} ) of the formulation,
wherein the training data for each reference formulation of a multitude of reference formulations comprise (i) a numerical representation (R_{F}) of the reference formulation and (ii) at least one measured stability feature (S_{M}), where the at least one measured stability feature (S_{M}) is a measure of the stability of a system comprising the reference formulation and at least one material in contact with the reference formulation,
wherein the training of the machine learning model (MLM) for each reference formulation comprises:
inputting the numerical representation (R_{F}) of the reference formulation into the machine learning model (MLM); inputting the numerical representation (R_{F}) of the reference formulation into the machine learning model (MLM);
receiving at least one predicted stability feature (S_{P}) from the machine learning model (MLM); receiving at least one predicted stability feature (S_{P}) from the machine learning model (MLM);
quantifying a variance between the at least one measured stability feature (S_{M}) and the at least one predicted stability feature (S_{P}); and
modifying the model parameters with regard to reducing the variance;
receiving a recipe (R*) of a test formulation;
ascertaining constituents of the test formulation on the basis of the recipe (R*);
ascertaining physical and/or chemical properties of the ascertained constituents;
creating a numerical representation (R_{F}*) of the test formulation, where the numerical representation (R_{F}*) of the test formulation comprises a numerical representation (R_{I1}*, R_{I2}*, R_{I3}*) of the ascertained constituent for each ascertained constituent, where the numerical representation (R_{I1}*, R_{I2}*, R_{I3}*) of each ascertained constituent comprises a value for the amount of the constituent in the test formulation and values for the ascertained physical and/or chemical properties of the constituent;
inputting the numerical representation (R_{F}*) of the test formulation into the trained machine learning model (MLM^{T});
receiving at least one predicted stability feature (S_{P}*) from the trained machine learning model (MLM^{T}); and
outputting and/or storing the at least one predicted stability feature (S_{P}*) and/or transmitting the at least one predicted stability feature (S_{P}*) to a separate computer system.
